(19) United States
(12) Patent Application Publication
(10) Pub. No.: US 2003/0091617 A1
(43) Pub. Date: May 15, 2003

(54) Gel-coated oil absorbing skin wipes

(76) Inventors: James S. Mrozinski, Oakdale, MN (US); Jayshree Seth, Woodbury, MN (US); James E. Thorson, Hudson, WI (US)

Correspondence Address: Office of Intellectual Property Counsel, 3M Innovative Properties Company, PO Box 33427, St. Paul, MN, 55133-3427, US

(21) Appl. No.: 09/876,704

(22) Filed: Jun. 7, 2001

(51) Int. Cl. A61K 9/70

(52) U.S. Cl. 424/443

(57) ABSTRACT

An oil absorbing wipe material suitable for wiping a users skin comprising an oil absorbing porous substrate having a transparency of less than 65 percent which porous substrate changes transparency upon absorption of facial oil, said porous substrate having a nontacky flexible coating on at least a portion of at least one face said coating comprising a film forming polymer with at least one additional additive which coating is visible on the coated face of the porous substrate and which coating does not penetrate to the opposite face of the porous substrate.

### BACKGROUND OF THE INVENTION

This invention relates to oil absorbent skin wipe products. The invention particularly relates to oil absorbent skin wiping products with additional functional layers.

A significant amount of oil continuously oozes out of the skin of the face, particularly the nose, cheek, forehead and middle forehead. To maintain cleanliness and to improve the spreadability of cosmetics it is important to remove any excess oil or sebum. Soap and water work to some extent but there are always times when one is not able to wash. Dry methods of removing these facial oils include the use of thin oil absorbent wipe materials. Oil absorbing wipes for removing facial oil have also been described in the art. These wipes generally must be thin, conformable and non-abrasive, considerations not relevant to industrial oil absorbent materials.

Conventional paper type wipes have been used to remove facial oil. For example, natural or synthetic papers using vegetable fibers, synthetic pulp or kenaf have been used. These oil absorbent papers however are often irritating to the skin due to the hard and stiff nature of the fibers. To improve their smoothness, these papers have been calendered and/or coated with powders such as calcium carbonate and sizing agents. Calendering however is not necessarily permanent and surface fibers can reform into a rough surface unless substantial amounts of binder or sizing agents are used, which decrease oil absorption. Paper wipes are also poor indicators as to their effectiveness as papers generally do not significantly change appearance when they have absorbed oil or sebum.

Improvements to oil absorbing papers are described in Japanese Kokai No. 4-45591 which teaches adhering porous spherical beads onto the surface of an oil absorbing paper so as to solve the problems caused by calendering or coating of paper with powders such as calcium carbonate powders. These beads also are used to allegedly increase the capacity of the papers to absorb sebum. Japanese Unexamined Patent Publication (Kokai) No. 6-319664 discloses a high-density oil absorbing paper produced by mixing (a) a pulp material containing vegetable fibers, as the main component with (b) an inorganic filler, followed by paper-making to form a paper with a basis weight of 0.7 (g/cm²) or more. However, the oil absorbing papers disclosed in these patent publications still have a limited capacity to absorb oil or sebum and little indicating function as there is little change in opacity or color in the paper when oil is absorbed. Difficulty in confirming oil means that users of the oil clearing paper can not evaluate if or how much sebum is removed from the users' face when using the oil absorbing paper such that makeup can be applied with confidence.

An oil absorbing paper for sebum is also disclosed in Japanese Examined Patent Publication (Kokoku) No. 56-8606, or U.S. Pat. No. 4,643,939, which describes a cosmetic oil absorbing paper produced by mixing hemp fibers with 10 to 70% by weight of polyolefin resin fibers and making a paper with a basis weight of from 12 to 50 (g/cm²). This paper will allegedly clear upon absorption of oil but still requires conventional papermaking techniques and would be rough to the touch. Japanese Unexamined Utility Model Publication (Kokai) No. 5-18392, discloses an oil absorbing synthetic paper comprising an oil absorbing paper with a smooth surface coating of inorganic or organic powder material such as clay particles, silica fine-particles, and powdered fibers. These oil-absorbing papers allegedly have some oil indicating effect by clarifying the paper upon oil absorption thus confirming oil absorption. However, the powder coating lowers the oil absorption capacity for these papers and it is still difficult to attain a clear change in the appearance of this type of oil clearing paper after oil absorption.

Oil-absorbing webs produced by using thermoplastic fibrous material in place of cellulosic fibrous papers are known. Further, Japanese Unexamined Patent Publication (Kokai) No. 9-335451 (WO99/29220) discloses an oil wipe made of a porous thermoplastic film. This oil absorbing wipe film has higher oil absorption capacity than the oil absorbing papers and is also superior in confirming removal of oil following wiping as compared to oil absorbing papers. It is believed that the reason for this good oil removal indicating functionality is that these porous thermoplastic films exhibit low light transmittance before oil absorption because of irregular reflection of light, but the light transmittance increases substantially after the micro-pores of the film are filled with oils producing a large change in the film's opacity or light transmittance, and therefore appearance. This change in opacity clearly confirms to the user the removal of oil or sebum from his or her skin. U.S. Pat. No. 4,532,937 to Miller describes analytical film for collecting sebum as it is secreted from the sebaceous glands of a subject comprising an open-celled, microporous and hydrophobic polymeric film, and a fibrous material having coated on one major surface a layer of synthetic, pressure-sensitive adhesive consisting essentially of high molecular weight components. The Miller patent describes its material as having pores of such a size and distribution that the film is opaque or opalescent when the pores are empty or filed with air but can become translucent or transparent upon absorption of a liquid such as sebum. However, the very small pores described for this film or material (less than 0.1 microns) do not provide a material best suited for use in cosmetic applications due to the slow oil absorption rates.

It is an object of the invention to form an oil absorbing wipe having a clear oil indicating function, such as described in WO99/29220, which can also deliver other agents or treatments to the skin following oil removal which product is easy to directly manufacture. Further, it is desirable that these additional agents are clearly visible on the oil absorbing wipe so as to inform the user as to which side of the wipe to use to remove facial oil and which side of the wipe to use to apply or use the additional skin treatment.

### BRIEF SUMMARY OF THE INVENTION

The invention is directed at an oil absorbing wipe material for wiping a users skin. The wipe comprises an oil absorbing porous substrate generally having a transparency of less than 65 percent which porous substrate changes transparency by at least 30 percentage points when loaded with a relatively low level of oil as found on an individual's face. The porous substrate has a nontacky flexible coating on at least a portion of at least one face. The coating comprises a film forming polymer with at least one additional additive which coating is visible on the coated face of the porous substrate and which coating does not penetrate to the opposite face of the porous substrate. Generally, the film forming polymer coating penetrates from 10 to 90 percent of the thickness of the oil absorbing porous substrate and the additional ingredient is an active or skin modifying agent.

### BRIEF DESCRIPTION OF THE DRAWINGS

FIG. 1 is a schematic diagram of an apparatus suitable for use in forming the invention wipes.

FIG. 2 is a perspective view of a dispensable package of oil absorbing wipes.

FIG. 3 is a perspective view of a dispensable package of oil absorbing wipes according to a second embodiment.

FIG. 4 is a perspective view of a dispensable package of oil absorbing wipes according to a third embodiment.

FIG. 5 is a perspective view of a dispensable package of oil absorbing wipes according to a fourth embodiment.

FIG. 6 is a side view photomicrograph of counter example C7 showing the penetration of a brittle film forming coating into an oil absorbing wipe.

FIG. 7 is a side view photomicrograph of example 2 showing the penetration of a flexible film forming coating into an oil absorbing wipe.

### DETAILED DESCRIPTION

The present invention oil absorbent wipe in a porous filmlike thermoplastic material which in a first preferred embodiment is generally a porous stretched or oriented film made of a thermoplastic material or alternatively in a second preferred embodiment a consolidated porous nonwoven fiber web which is filmlike. Filmlike as used herein is defined as thermoplastic films or consolidated nonwovens of fibers. In a less preferred embodiment, conventional paperlike oil absorbent wipes can be coated with the invention film forming polymer layer. The wipe will be coated on at least a portion of one face or side with a film forming polymer generally having an additional useful ingredient within the film forming polymer layer.

The porosity of the interstitial volume per unit area of the porous film material of the oil absorbent wipe of the first preferred embodiment is preferably in the range of 0.0001-0.005 cm³ as calculated by the equation:

Interstitial volume per unit area=[film thickness (cm)×1 (cm)×1 (cm)×void content (%)]/100 (where the void content is the percentage of voids in the porous film).

The “void content” is more specifically defined as the percentage of an amount of filling material, when all of the voids of the porous film are filled with a material of the same composition as the film, with respect to a film with no corresponding voids. The void content of the porous film is preferably in the range of 5-50% and the thickness is preferably in the range of 5-200 μm.

The porous stretched film may be produced by various different methods using a thermoplastic material as the starting substance. In one preferred method, the film is produced by adding a filler to a transparent crystalline thermoplastic resin, forming a film using conventional methods such as blown extrusion or casting, and then stretching the film to create fine voids therein. A porous stretched thermoplastic film obtained in this manner has a large percentage of voids constituting the volume of the wipe compared to conventional paper oil cleaning wipes, and has excellent absorption of skin oils per unit area. Also, since the thermoplastic film has a structure with a uniform distribution of many fine voids, prior to wiping of skin oils from the skin surface it appears non-transparent due to light dispersion by the pore structures. However, after oil absorption the oils fill the voids or pores thus either preventing or reducing the degree of light dispersion. This together with the original opaque or transparent nature of the thermoplastic forming the film allows the oil absorbing effect to be clearly assessed by a change in transparency or opacity.

Examples of transparent crystalline thermoplastic resins which can be used as the film forming material for production of the porous unstretched thermoplastic film of the invention include, but are not limited to, polyethylene, polypropylene, polybutylene, poly-4-methylpentene and ethylene-propylene block copolymer.

Examples of preferred nonparticulate fillers that can be used in combination with the aforementioned thermoplastic resins to provide the fine voids include, but are not limited to, mineral oils, petroleum jelly, low molecular weight polyethylene, soft Carbowax and mixtures thereof. These nonparticulate fillers are preferred as they exhibit transparency upon absorption of oil. Mineral oils are preferred among these fillers because of their relatively low cost. However, additionally conventional particulate based fillers can also be used to form the porous film, such as talc, calcium carbonate, titanium dioxide, barium sulfate, etc.

The aforementioned fillers can be varied within a wide range within the starting thermoplastic resin used for production of the film. The amount of filler used is preferably in the range of 20-60% by weight, and more preferably 25-40% by weight of the starting thermoplastic material. If the amount of filler added to the starting material is under 20% by weight, the void content of the film resulting after stretching is reduced, thus lowering the amount of oil absorption, while if it is above 60% by weight it becomes more difficult to produce flexible coherent films.

Other additives may also be added as necessary in addition to the thermoplastic resin and filler in the production of the porous stretched thermoplastic film. For example, organic acids such as carboxylic acid, sulfonic acid and phosphonic acid, and organic alcohols. As additional suitable additives there may also be mentioned, for example, inorganic and organic pigment, aromatic agents, surfactants, antistatic agents, nucleating agents and the like.

The main starting materials and optional additives are melted and/or combined to form a film, producing a filler-containing thermoplastic film. The melting and mixing step(s) and the subsequent film forming step may be carried out according to known methods. An example of a suitable melt mixing method is kneading with a kneader, and examples of suitable film forming methods are the blown film method and the casting method. The blown film method, for example, can give tube-shaped films by melt mixing the main starting material, etc. and then blowing it up from a circular die. The casting method can give films by melt mixing the main starting material, etc. and then extruding it from a die onto a smooth or patterned chilled roll (cold roll). In a modified form of this casting method, the nonparticulate additives and/or fillers may be removed by washing off or extracting with a suitable solvent after extrusion of the melted mixture onto the chilled roll.

The formed thermoplastic film is then stretched to provide it with fine voids. As with the film forming, the stretching may also be carried out according to known methods, such as uniaxial stretching or biaxial stretching. For example, in the case of biaxial stretching, the stretching in the lengthwise direction may be accomplished by varying the speed of the driving roll, and the stretching in the widthwise direction may be accomplished by mechanical pulling in the widthwise direction while holding both sides of the film with clips or clamps.

The conditions for the film stretching are not particularly restricted, but the stretching is preferably carried out so as to give a void content in the range of 5-50% and a stretched film thickness in the range of 5-200 μm. If the void content upon stretching of the film is under 5% the amount of oil absorption will be reduced, while if it is over 50% the amount of oil absorption will be too great, making it difficult to clearly assess the oil absorbing effect. Also, if the film thickness is under 5 μm the amount of oil absorption capacity will be too low and the film will tend to adhere to the face making it more difficult to handle, while if it is over 200 μm the amount of oil absorption capacity will be too great and the film may feel stiff and harsh against the user's skin.

The stretching ratio for the thermoplastic film is usually preferred to be in the range of 1.5 to 3.0. If the stretching ratio is under 1.5 it becomes difficult to achieve a sufficient void content for oil absorption, while if it is over 3.0 the void content could become too large, causing too much oil absorption.

The average size of the voids formed by stretching of the film is usually preferred to be in the range of 0.2 to 5 μm. If the void size is under 0.2 μm it becomes impossible to rapidly absorb enough skin oil to create a clear change in transparency, while if it is over 5 μm the amount of oil absorption needed to permit a visible change in transparency may be too great.

As mentioned above, the interstitial volume per unit area of the porous stretched thermoplastic film obtained by the stretching process described earlier is preferably in the range of 0.0001-0.005 cm³, and more preferably in the range of 0.0002-0.001 cm³, as calculated by the equation defined above. If the interstitial volume of the film is under 0.001 cm³ it becomes difficult for the user to hold the oil cleaning wipe, while if it is over 0.005 cm³ the amount of oil absorption is too great, and it becomes difficult to clearly assess the oil absorbing effect.

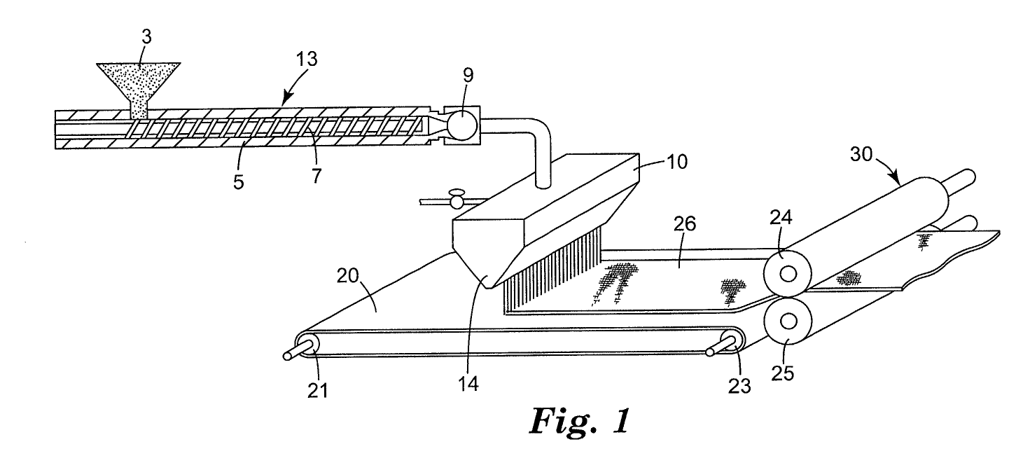

The second embodiment of a film-like porous wipe is a consolidated nonwoven web preferably formed of thermoplastic microfibers. A representative apparatus useful for preparing such a web or wipe product is shown schematically in FIG. 1. Part of the apparatus for forming blown fibers is described in Wente Van A., “Superfine Thermoplastic Fibers” in Industrial Engineering Chemistry, Vol. 48, p. 1342 et seq. (1956), or in Report No. 4364 of the Naval Research Laboratories, published May 25, 1954, entitled “Manufacture of Superfine Organic Fibers”, by Wente, V. A.; Boone, C. D.; and Fluharty, E. L. Modifications to this basic design are discussed in U.S. Pat. Nos. 4,818,463; 3,825,379; 4,907,174 and 4,986,743. This portion of the illustrated apparatus comprises a die 10, which has a set of aligned side-by-side parallel die orifices 14. The die orifices 14 open from a central die cavity. Typically, the diameter of the orifices will be on the order of from about 250 microns to about 500 microns. From about 2 to about 20 such orifices will be provided per linear centimeter of die face. Typically, the length of the orifices will be from about 1 mm to about 5 mm. The polymer is introduced to the die orifices 14 and the central die cavity from a melt extruder 13 having a resin hopper 3, a barrel 5, and a screw 7 inside the barrel 5. The molten polyolefin resin exits from the extruder barrel 5 into a gear melt pump 9 which permits improved control over the flow of the molten polymer through the downstream components of the apparatus. Upon exiting from the pump 9, the molten resin flows into a die 10 containing the die cavity through which liquefied fiber-forming material is advanced. The fiber forming thermoplastic polymer is extruded from the die orifices 14 into an attenuating airstream of heated air. This attenuating airstream is maintained at high velocities and exits from orifices or slots on either side of the set of die orifices 14. The high-velocity air is supplied to slots from two peripheral cavities. The heated air is generally about the temperature of the polymer melt or higher (e.g., 20 to 30° C. above the melt temperature).

The fibers exiting from the die orifices are attenuated by the high velocity heated air from slots and are collected on collector 20, such as a belt, at a distance a from the die. The distance a is generally from 10 to 25 cm with different preferred regions for different polymers depending on the crystalline behavior of the polymer, how rapidly it is quenched to a totally non-tacky condition or other process conditions. The collector can be a flat screen, a drum, a cylinder or a finely perforated screen belt 20 as shown in FIG. 1. Cylinders 21 and 23 drive the belt 20. A gas-withdrawal device can be located behind perforated collectors to facilitate collection of the fibers, on the screen or other perforated collector surface, as a web 26. From the collector 20, the web 26 is taken to a calender 30 where the web is consolidated under pressure, preferably from 500 to 1600 Newtons per lineal centimeter. This consolidation is advantageously carried out by calendering in the nip between two generally smooth rolls 24 and 25 (e.g., they contact each other over about 90 percent of their surface area or greater, preferably 99 percent or greater), having a Shore A durometer hardness of about 50 or more, although one roll preferably has a Shore A durometer hardness of less than about 95. The consolidated web can then be collected and subsequently converted into individual wipes.

The webs are formed of fiber-forming thermoplastic materials, which materials include, for example, polyolefins, such as polyethylene, polypropylene or polybutylene; polyesters, such as polyethylene terephthalate or polybutylene terephthalate; polyurethanes or polyamides such as nylon 6 or nylon 66. The microfibers have an average diameter of less than 10 micrometers, preferably with an average diameter of 7 micrometers or less. Smaller average fiber diameters may be obtained with smaller diameter orifices and/or by decreasing the polymer flow rate or by increasing gas withdrawal behind the collecter.

The oil absorbing wipes are formed from the consolidated film-like fiber nonwoven webs are such that the wipe generally has a void volume of from 40 to 80 percent, preferably 45 to 75 percent and most preferably 50 to 70 percent. Where the void volume is greater than 70 percent it is difficult to obtain a rapid change in transparency or opacity as large amounts of oil are necessary to create this change, also the material becomes to compliant and difficult to handle. Where the void volume is less than 40%, the material becomes too stiff and has an insufficient capacity to absorb oil. The average pore size of the wipe is generally from 3-15 microns, preferably 3 to 12 microns and most preferably 4 to 8 microns. If the pore size is less than 3 microns, it is difficult to get the rapid oil absorption rate needed. Void volume and pore size generally can be decreased by higher consolidation conditions and/or decreasing the average fiber diameter or narrowing the range of fiber diameters. If the pore size is greater than 15 microns the ability to retain absorbed oil is lessened as is the rapid oil indicating function. Generally the void volume, basis weight and pore size should be provided to yield an oil absorption capacity of from 0.7 to 6 mg/cm², preferably 0.8 to 5 mg/cm² and most preferably 0.9 to 4 mg/cm². If the oil absorption is less than this then the capacity to absorb facial oil is insufficient for most users and when greater than these levels then the rapid oil absorption indicating function is adversely impacted for most users.

A preferred thermoplastic material for forming the web fibers is polypropylene wherein the desired initial and end opacity for a given wipe is controlled by the basis weight of the web forming the wipe material, the hardness of the calendering rolls, and the calendering (or consolidation) pressure and temperature. Generally, for polypropylene, a web or wipe basis weight of about 10 gm/m² to 40 gm/m² has been found suitable to provide an adequate initial transparency while allowing a change in transparency at a suitably low oil loading level with a relatively soft hand. Generally, the Hand of the wipe should be 8 grams or less, preferably 1-7 grams and most preferably 1-6 grams. For polypropylene wipes, basis weights of greater than about 40 gm/m² are too stiff to be useful as a facial wipe. For fibers formed of other polymers or polymer blends under similar calendering conditions, different wipe basis weight ranges may be suitable depending on the oil absorbing properties and relative stiffness of the fibers forming the web.

Higher calendering temperatures and pressures have been found to have significant effects on the original transparency, pore size and void volume and also the resulting oil absorption capacity of the consolidated wipe. Higher calendering temperatures in particular significantly increase the original transparency, thus decreasing the oil-indicating value of the wipe. Under certain circumstances, it would be desirable to use chilled calendering rolls to counteract this effect. However, when a web is over-calendered (e.g., under too high a pressure and/or temperature), the web does not become more rigid, however, the oil indicating function and absorption capacity does decrease.

If the original opacity is inadequate to produce a significant enough change in opacity, opacifying agents such as silica, talc, calcium carbonate or other like inorganic powders can be used at low levels. Such powders could be coated on the surface of the wipes or incorporated into the web structures. Suitable methods for incorporating opacifying agents into the web include that taught in U.S. Pat. No. 3,971,373 where a stream of particles is entrained into two separate converging melt-blown microfiber streams prior to collection. Another method of incorporating particulates is taught in U.S. Pat. No. 4,755,178 where particles are introduced into an airstream that converges into a flow of melt-blown microfibers. Preferably, only a small amount of such opacifying agents are included as they have the tendency to detract from the wipe softness.

In addition to the above, other conventional web additives such as surfactants, colorants, and antistatic agents can be incorporated into the web by known methods.

The invention oil absorbent wipes are generally characterized by the ability to change from opaque to translucent after absorbing only a moderate amount of oil, such as would be present on a person's skin (e.g., from 0 to 8 mg/cm²). The oil absorbent wipes are particularly useful as cosmetic wipes as after absorbing skin oil at the levels excreted from common sebaceous glands, they will turn translucent, thus indicating that the undesirable oil has been removed and that makeup or other skin treatments can be applied. The oil-indicating effect is provided by an oil absorbing wipe having an initial transparency of about 65 percent or less, preferably 60 percent or less with an ability to change transparency by about 30 percentage points or more, preferably 35 percentage points or more with a relatively low level of oil loading (e.g., 6 mg/cm²). The oil absorbing wipe is generally used as a single layer material but could be laminated to other like web materials, or films or the like.

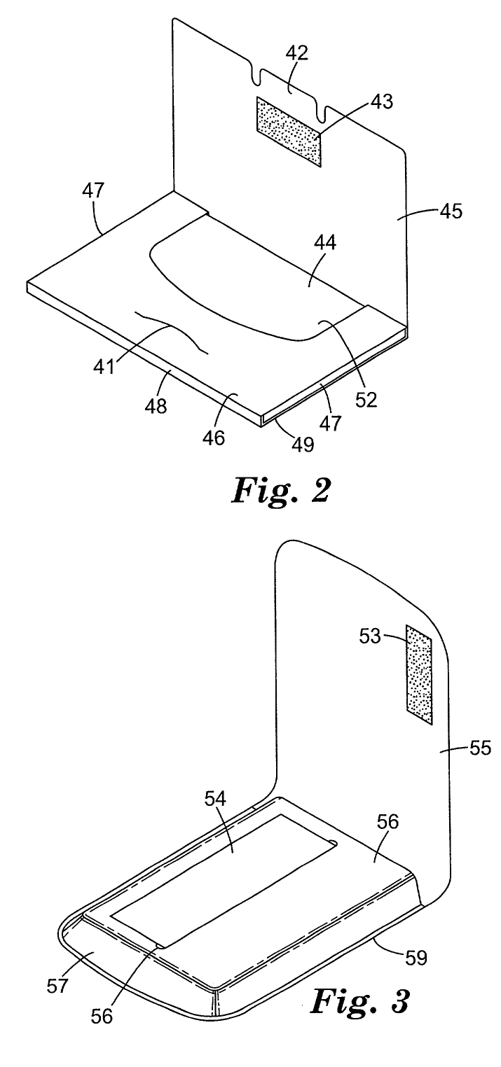

Referring to FIG. 2, a dispensable package of oil wipes in accordance with the invention comprises a dispensable package 40 including individual wipes 44 of oil absorbent wipe material. The package 44 generally comprises a top wall 46 and bottom wall 49, generally parallel to one another, and two side walls 47. A front edge 48 is provided where the back edge is formed into a flap 45, which can be folded down onto the upper face 46 of the package 40. The flap 45 can engage with the package 40 by use of an adhesive or the like, provided as is known in the art. Alternatively, a tab 42 engagable within a slot 41 can be used as a macro-mechanical type closure. Other conventional methods known in the art include the use of cohesive materials, hook and loop fasteners, living hinges, snaps and the like to keep the flap 45 in place to cover the access opening 52 to the wipes. The dispensable package 40 contains an access opening 52 which permits a user to grasp an individual wipe and withdraw it from the package 40 for use. Generally, the access opening 52 is at its largest dimension smaller than the largest length or width dimension of the dispensable oil absorbing wipe material or wipe. However, if the individual wipes are connected in a manner that they are separable from one another then the access opening should be as large or smaller than the dimension of the wipe which is pulled through the access opening.

The discrete wipe materials can be either separated from one another or separable from one or another, both are considered to be discrete wipes or wipes according to the invention. Generally, separable wipes are provided by having a frangible connection between the discrete wipes which allow the user to break and to separate the discrete wipes one from the other. Frangible connections can be created by lines of weaknesses such as perforations, score lines or by the use of additional weak adhesive-type attachment materials or by simply frictional engagement. Discrete separate wipes would require no breaking of a frangible connection. The wipes further can be stacked, provided in a roll, or folded and the like as is conventionally known for tissue-type papers. Folding is generally provided by an interleaving arrangement via v-folds, z-folds or the like. With this type of folding, opposing overlapping ends of adjacent wipes allow removal of an upper wipe to provide the lower wipe in an engagable form by frictionally pulling the lower wipe up and out through an access opening for subsequent use.

An alternative embodiment of a dispensable package arrangement is shown in FIG. 3, the top wall portion 56 is provided with an access opening slot 54 through which a wipe of oil absorbent wipe material is graspable. In this embodiment, the discrete wipes of wipe material must be interconnected so that the upper wipe can pull the lower wipe up and through the opening 56. This interconnection can be by separate wipes that are folded in an interleaving manner as described above. Alternatively the wipes could be separable wipes as described above; for example; separable wipes can be interconnected through a frangible connection. The movable flap 55 is provided on a sidewall portion and, like the flap in the FIG. 2 embodiment, can be provided with a suitable closure element 53, such as a patch of pressure-sensitive adhesive.

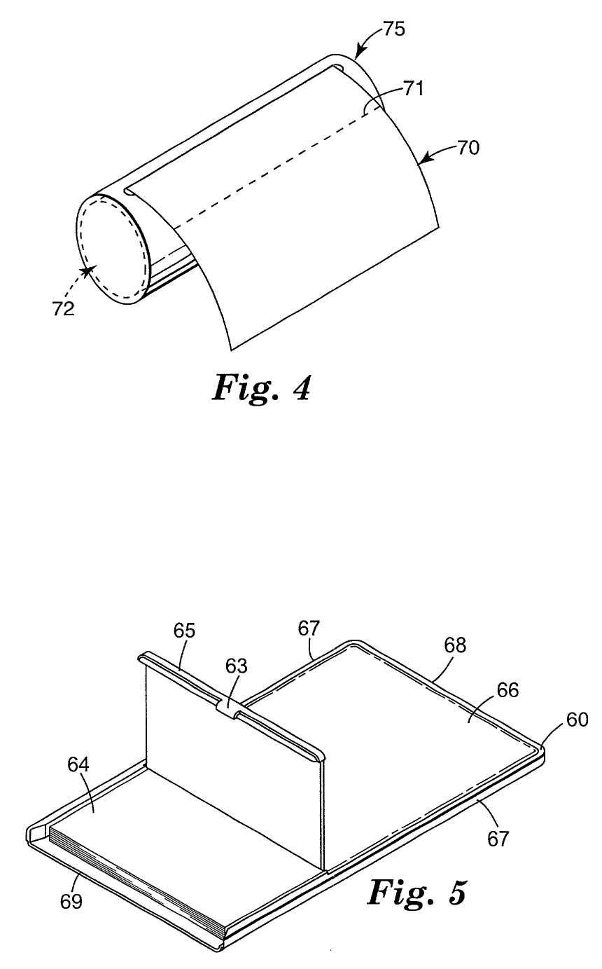

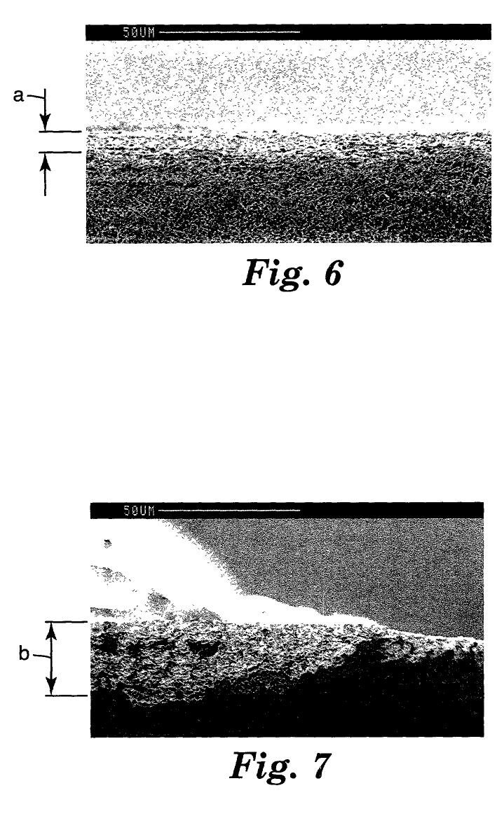

A further alternative embodiment of the dispensable oil absorbent wipe package is shown in FIG. 4 which shows a roll of discrete wipe materials 70 connected by frangible connections 71 which can be rolled into a roll form 72, with or without a core, allowing the materials to be grasped and dispensed from a roll dispenser 75.

FIG. 5 shows an alternative embodiment of a dispensable package of the oil absorbent wipes formed with a rigid frame container 60, preferably thermoplastic. The individual wipe materials 64 are contained within the container 60, which has a top wall 66 containing a movable flap 65, which is generally movable by a living hinge. A clasp 63 is provided on the outermost end of flap 65, which clasp 63 engages with the bottom wall 69 to provide for closure of the container 60. Side walls 67 contain the wipes 64 within the container 60 coupled with the upper walls 66 and lower wall 69. End wall 68 is preferably closed. In this embodiment, the individual wipes of discrete oil absorbent material would generally be stacked as separate wipes in an overlying stack preferably of coextensive wipes. The user would grasp an individual wipe and remove each one separately from the container using the frictional force of their fingers to separate the upper wipe from the immediate lower wipe. The individual wipes would then be used to remove skin oil by wiping over the user's face. Following use, the wipe is easily compacted into a small volume shape for easy disposal.

The individual discrete wipes can be of any suitable size, however, generally for most applications the wipes would have an overall surface area of from 10 to 100 cm², preferably from 20 to 50 cm². As such, the wipes would be of a size suitable for insertion in a package, which could easily be placed in the user's purse or pocket. The material forming the dispensable containers is generally not of importance and can be formed of suitable papers, thermoplastics, paper film laminates and the like. The shape of the tissues is generally rectangular; however, other suitable shapes such as oval, circular or the like can be used.

The oil-absorbing wipes of the invention can be coated with any active or nonactive ingredients or agents in the film forming polymeric coating. Additional ingredients can comprises a wide range of optional ingredients. Particularly useful are various active ingredients useful for delivering various benefits to the skin or hair during and after oil removal and cleansing.

The film forming coating compositions of the present invention can include a safe and effective amount of one or more pharmaceutically-acceptable active or skin modifying ingredients. The term “safe and effective amount” as used herein, means an amount of an active ingredient high enough to modify the conditions to be treated or to deliver the desired skin benefit, but low enough to avoid serious side effects. What is a safe and effective amount of the active ingredient will vary with the specific active ingredient, the ability of the active ingredient to penetrate through the skin, the age, health condition, and skin condition of the user, and other like factors.

The active ingredients useful herein can be categorized by their therapeutic benefit or their postulated mode of action. However, it is to be understood that the active ingredients useful herein can in some instances provide more than one therapeutic benefit or operate via more than one mode of action. The following active ingredients are useful in the compositions of the present invention. Anti-Acne Actives: examples of useful anti-acne actives include the keratolytics such as salicylic acid (o-hydroxybenzoic acid), derivatives of salicylic acid, retinoids such as retinoic acid and its derivatives (e.g., cis and trans); sulfur-containing D and L amino acids and their derivatives and salts, lipoic acid; antibiotics and antimicrobials; sebostats such as flavonoids; and bile salts such as scymnol sulfate and its derivatives, deoxycholate, and cholate. Anti-Wrinkle and Anti-Skin Atrophy Actives: examples of antiwrinkle and anti-skin atrophy actives include retinoic acid and its derivatives (e.g., cis and trans); retinol; retinyl esters; niacinamide, salicylic acid and derivatives thereof, sulfur-containing D and L amino acids and their derivatives and salts, thiols, hydroxy acids phytic acid, lipoic acid;lysophosphatidic acid, and skin peel agents (e.g., phenol and the like). Non-Steroidal Anti-Inflammatory Actives (NSAIDS): examples of NSAIDS include the following, propionic acid derivatives; acetic acid derivatives; fenamic acid derivatives; biphenylcarboxylic acid derivatives; and oxicams. Topical Anesthetics; examples of topical anesthetic drugs include benzocaine, lidocaine, bupivacaine, chlorprocaine, dibucaine, etidocaine, mepivacaine, tetracaine, dyclonine, hexylcaine, procaine, cocaine, ketamine, pramoxine, phenol, and pharmaceutically acceptable salts thereof. Artificial Tanning Agents and Accelerators; examples of artificial tanning agents and accelerators include dihydroxyacetaone, tyrosine, tyrosine esters such as ethyl tyrosinate, and phospho-DOPA. Sunscreen Actives; examples of sunscreens which are useful in the compositions of the present invention are those selected from the group consisting of 2-ethylhexyl p-methoxycinnamate, 2-ethylhexyl N,N-dimethyl-p-aminobenzoate, p-aminobenzoic acid, 2-phenylbenzimidazole-5-sulfonic acid, octocrylene, oxybenzone, homomenthyl salicylate, octyl salicylate, 4,4′-methoxy-t-butyldibenzoylmethane, 4-isopropyl dibenzoylmethane, 3-benzylidene camphor, 3-(4-methylbenzylidene) camphor, titanium dioxide, zinc oxide, silica, iron oxide and mixtures thereof. Other known active agents such as antibiotics or antiseptics may also be used.

The coating compositions of the present invention can comprise a wide range of other components which can provide skin benefits, or modify the skin or modify the coating composition. These additional components should generally be pharmaceutically acceptable. The CTFA Cosmetic Ingredient Handbook, Second Edition, 1992, which is incorporated by reference herein in its entirety, describes a wide variety of nonlimiting cosmetic and pharmaceutical ingredients commonly used in the skin care industry, which may be suitable for use in the coating compositions of the present invention. Nonlimiting examples of classes of ingredients are described at page 537 of this reference. Examples of these and other classes include: fillers, abrasives, absorbents, anticaking agents, antioxidants, vitamins, binders, biological additives, buffering agents, bulking agents, chelating agents, chemical additives, colorants, cosmetic astringents, cosmetic biocides, denaturants, drug astringents, external analgesics, film formers, fragrance components, humectants, opacifying agents, pH adjusters, preservatives, propellants, reducing agents, skin bleaching agents, and sunscreening agents. Preferred are particulate additives that can be used to act as opacifiers, pigments or fillers. Suitable inorganic pigments or fillers would be talc, titanium dioxide, calcium carbonate and zinc oxide.

Also useful as additives are aesthetic components such as fragrances, pigments, colorings, essential oils, skin sensates, astringents, skin soothing agents, and skin healing agents.

Suitable preferred film forming agents for forming the film forming coating include film-forming polymers such as polyvinyl alcohol, dimethicone copolyol, poly(N-vinyl formamide), polyacrylamide, poly(hydroxyethyl), methacrylate polyethylene oxide, polyvinyl pyrrolidone, polyvinyl acetate, polyhydroxylalkyl cellulose ethers such as carboxymethylcellulose, 2-hydroxyethyl cellulose, 2-hydroxyethylmethyl cellulose or 2-hydroxypropyl cellulose.

Water insoluble film forming polymers suitable for this invention include, but are not limited to, water insoluble polyamide polymers; esters of polymeric carboxylic acids, e.g., polyacrylate polymers; polypropylene oxide and derivatives thereof; and the like.

Water soluble film-forming polymers may be cationic, anionic or nonionic polymers. Preferred to water soluble film forming polymers include: cellulose derivatives such as quaternary nitrogen-containing cellulose ethers, hydroxyethyl cellulose, hydroxypropyl cellulose and hydroxyethyl alkali metal carboxylalkyl cellulose derivatives, and free acid hydroxyalkyl carboxyalkyl cellulose derivatives; polyvinyl alcohol; vinylpyrrolidone homopolymers and copolymers; polycarboxylic acid derivatives; polyacrylamides; vinyl methyl ether homopolymers and copolymers; ethylene oxide resins; and the like.

The choice of film-forming polymer is not critical and may comprise any of the above types of water soluble or water insoluble film-forming polymers or blends thereof. However, the film-forming polymer when applied to the oil absorbent wipe must be in solution with an evaporative solvent to provide for effective penetration of the coating into the oil-absorbing wipes such that the film forming polymer is at least partially penetrated into the wipe when the evaporative solvent is removed. The film-forming polymer may be completely or partially soluble in the solvent and the solvent must at least in part wet the oil absorbent wipe. However, if the film-forming polymer is only partially soluble, the concentration of the film-forming polymer should be below the saturation level so that all the film-forming polymer is at least partially soluble in the solvent, the saturation level must also be sufficient, i.e., high enough, to provide at least the minimum weight ratio of polymer to other active agents or ingredients. The viscosity of the film forming polymer coating or solution is generally at least 10 and from 2000 to 10,000 for coating porous film substrates, preferably 3000 to 50,000. With consolidated nonwoven type substrates the viscosity of the of the film forming polymer coating is generally from 10, 000 to 100,000 cps and preferably 15,000 to 50,000. The use of too low a viscosity solution would cause excessive penetration into the porous wipe material making the coating visible from the opposite face. Too high a viscosity can result in too low a bond between the coating and the oil absorbent wipe material resulting in the coating falling off or cracking and increasing the difficulty in coating. The percent solids in the coating solution is generally 50 to 80, preferably 60 to 70 for porous film substrates with consolidated nonwoven substrate the percent solids is generally 50 to 80, preferably 65 to 75. The viscosity and percent solids is adjusted to the desired levels by use of the solvent, fillers or viscosity modifying agents. The choice of solvent and percent solids can be used to adjust the viscosity and penetration of the solution into the porous wipe.

The amount of particulates in the solids is adjusted depending on the film forming polymer. The dried coating following evaporation of the solvents and other volatile components is generally a film forming polymer with about 35 to 55 percent by weight particulate filler to film forming polymers and other nonparticulate solids in the coating, preferably 40 to 50 percent particulate filler to other solids in the coating. Additional active agents or other ingredients make up the remainder of the dried coating. This generally provides a nontacky but flexible or nonbriffle film depending on the nature of the film forming polymer. The exact amount of filler useable with a particular film forming polymer depends on the nature of the film former and the size of the particulate filler. Generally, the particulate filler is a solid having an average size of from 0.1 to 30 microns. If too low an amount of filler is used, the coating becomes tacky and causes adjacent wipes to block or adhere to each other in a package form. If too high an amount of filler is used, the coating becomes brittle and falls off to easily. If a coating falls off this would result in a non either no coating over a portion of the wipe that has been coated or a nonuniform distribution of the coating over the area of the oil absorbent wipe that has been coated. For functionality purposes, it is preferred that there is a substantially uniform distribution of a continuous or pattern coating over a substantial portion of one face of the wipe, generally 10 to 100 percent of one face of the wipe, preferably 50 to 100 percent. If the coating is a discontinuous pattern it can be applied in any suitable pattern such as dots, lines, discrete patterns, logos, etc., as would be known in the art.

Generally, the penetration of the film forming polymer coating into the oil absorbent wipe is less than the thickness of the wipe, preferably from 10 to 90 percent of the wipe thickness and most preferably from 20 to 80 percent of the wipe thickness. If the percent penetration is too high, then the user will not be able to differentiate the oil absorbent side from the polymer coated side of the wipe. Further, too high a degree of penetration of the film forming polymer into the wipe will significantly impair the oil absorbency functionality of the oil absorbent wipe. If the film forming polymer has little or no penetration into the wipe then the coating can easily be removed or fall off the wipe surface.

The evaporative solvent used with the film forming polymer may be any liquid in which the film-forming polymer is completely or substantially soluble and which will readily evaporate when used in compositions of the present invention. The evaporation characteristics of the evaporative solvent can be usually be characterized based on the partial vapor pressure of such solvents. The partial pressure of evaporative solvents of the present invention will generally be greater than 1 preferably from about 10 mm to about 250 mm at 25° C. The partial pressure of many compounds may be determined experimentally using standard procedures as reported in literature. Preferred solvents include alcohols and water.

Organic gelling agents can be employed as viscosity modifiers in amounts of from about 5% to 25% and comprise those of natural or synthetic origin. Preferred gelling agents are starches such as the glycerol starches, microcrystalline cellulose and hydroxyalkyl cellulose ethers such as hydroxypropylmethyl cellulose (HPMC), hydroxymethyl cellulose (HMC), carboxymethyl cellulose (CMC), 2-hydroxyethyl cellulose, 2-hydroxyethylmethyl cellulose, propylene carbonate and 2-hydroxypropyl cellulose (Klucel® H); or one or more short carbon chain alcohols such as ethanol, isopropyl alcohol, propylene glycol, butylene glycol, hexylene glycol, polyethylene glycol, methoxypolyethylene glycol and their derivatives.

One embodiment of a preferable method for manufacturing the wipe pack of the present invention is as follows. The film forming polymer with the active or inactive skin modifying substances or other additives and fillers are homogeneously stirred, and a solvent is added thereto to adjust the viscosity, to give a coatable polymer solution or slurry. In this case, it is preferable that the solvent content is 25% to 50% by weight. Thereafter, the film forming polymer coating solution or slurry is uniformly spread directly onto an oil absorbing wipe substrate with an applicator continuously or in a pattern, preferably without any intermediate layer such as a fibrous layer. The solvent is subsequently actively evaporated by use of heat reduced pressure or the like or allowed to evaporate in the air. The coated oil absorbing wipe substrate is then cut into discrete pieces or as a continuous piece with frangible connections and provided into a package as is described above.

### Test Methods

Viscosity

The viscosities of the coatings used for the facial wipes of the invention were measured using a Brookfield Viscometer. Spindle 2 was used at 1.5 RPM for all measurements except where noted. Results are reported in centipoise (cps).

Visual Appearance

A determination was made for each of the examples and counter-examples as to whether the coatings had soaked through the wipe and thus became visible from the uncoated side of the wipe as noted with a ‘Y’ (Yes) or an ‘N’ (No) in the following tables. Visibility of the coating from the uncoated side of the wipe is not desirable, as this may interfere with the users ability to detect sebum absorption from the wiping of their skin.

### EXAMPLES

### Examples 1-9

Multi-functional facial wipes were prepared using the coatings shown below in Table 1 (all parts by weight) and a microporous film similar to that described in PCT application WO 99/29220 Example 1 as a substrate, having the following composition: 5D45 polypropylene (62.9%, Union Carbide Co.), mineral oil (35.0%, white oil #31, Amoco Oil & Chemical Co.), iron oxide red pigment (2.0%, CI #77491 russet, Sun Chemical Co.) and Millad 3988 nucleating agent (0.1%, Milliken Chemical). The microporous film had a thickness of 37 microns and a void content of 30%. The coatings were prepared by weighing the listed ingredients followed by mixing and homogenizing by hand or mechanical shaker. The coatings were then diluted with isopropyl alcohol to various percent solids. For simplicity, all ingredients other than the isopropyl alcohol were considered solids. This same definition of percent solids is used for all purposes in the invention. The microporous films were coated using the following technique. A 7 cm×10 cm piece of microporous film was laid onto a flat surface. A thermoplastic screen constrained in an embroidery hoop, having 15-1 mm diameter perforations/cm², was placed over the microporous film. A modest quantity of the coating solution was placed onto the screen. The coating was forced through the screen openings with a thermoplastic scraper onto the microporous film, resulting in a dot coating. The coated microporous film was then allowed to air dry for 24 hours before making observations.

TABLE 1

| | Example | | | | | | | | |
|---|---|---|---|---|---|---|---|---|---|
| Component | 1 | 2 | 3 | 4 | 5 | 6 | 7 | 8 | 9 |
| PVP-1⁽¹⁾ | 46 | 46 | | | | | | | |
| PVP-2⁽²⁾ | | | 46 | 46 | 46 | 46 | | | |
| PVP-3⁽³⁾ | | | | | | | 46 | 46 | 46 |
| Butylene | 6 | 6 | 6 | 6 | 6 | 6 | 6 | 6 | 6 |
| glycol⁽⁴⁾ | | | | | | | | | |
| Talc⁽⁵⁾ | 46 | 46 | 46 | 46 | 46 | 46 | 46 | 46 | 46 |
| Salicylic | 2 | 2 | 2 | 2 | 2 | 2 | 2 | 2 | 2 |
| acid⁽⁶⁾ | | | | | | | | | |
| % Solids | 70 | 75 | 60 | 65 | 70 | 75 | 60 | 65 | 70 |
| Viscosity | 3500 | 22500 | 3500 | 13250 | 37750 | 274000⁽⁷⁾ | 3250 | 15750 | 37750 |
| % | 46 | 46 | 46 | 46 | 46 | 46 | 46 | 46 | 46 |
| Particulates | | | | | | | | | |
| after drying | | | | | | | | | |
| Did coating | N | N | N | N | N | N | N | N | N |
| soak thru to | | | | | | | | | |
| noncoated | | | | | | | | | |
| side? (Y/N) | | | | | | | | | |

⁽¹⁾polyvinylpyrrolidone - 10,000 Mw, Aldrich Chemical Co.
⁽²⁾polyvinylpyrrolidone - 40,000 Mw, Povidone K-30, BASF Chemical Co.
⁽³⁾polyvinylpyrrolidone - 55,000 Mw, Aldrich Chemical Co.
⁽⁴⁾butylene glycol - 1,3-butanediol, Aldrich Chemical Co.
⁽⁵⁾talc - Aldrich Chemical Co.
⁽⁶⁾salicylic acid - Aldrich Chemical Co.
⁽⁷⁾measured with spindle 4 at 1.5 RPM

### Examples 10-19

Multi-functional facial wipes were prepared as in Examples 1-9 above except several different glycols were used at various percent solids to demonstrate possible variations in coating compositions as shown in Table 2 below.

TABLE 2

| | Example | | | | | | | | | |
|---|---|---|---|---|---|---|---|---|---|---|
| Component | 10 | 11 | 12 | 13 | 14 | 15 | 16 | 17 | 18 | 19 |
| PVP-1 | 46 | 46 | 46 | 46 | 46 | 46 | 46 | 46 | 46 | 46 |
| PEG-1⁽¹⁾ | 6 | 6 | | | | | | | | |
| PEG-2⁽²⁾ | | | 6 | 6 | | | | | | |
| PEG-3⁽³⁾ | | | | | 6 | 6 | | | | |
| HG-4⁽⁴⁾ | | | | | | | 6 | 6 | 6 | |
| MMBG-1⁽⁵⁾ | | | | | | | | | | 6 |
| Talc | 46 | 46 | 46 | 46 | 46 | 46 | 46 | 46 | 46 | 46 |
| Salicylic | 2 | 2 | 2 | 2 | 2 | 2 | 2 | 2 | 2 | 2 |
| acid | | | | | | | | | | |
| % Solids | 70 | 75 | 65 | 75 | 70 | 75 | 65 | 70 | 75 | 75 |
| Viscosity | 3000 | 27750 | | 32250 | 9550 | 34750 | 2250 | 4000 | 23250 | 32750 |
| % | 46 | 46 | 46 | 46 | 46 | 46 | 46 | 46 | 46 | 46 |
| Particulates | | | | | | | | | | |
| after drying | | | | | | | | | | |
| did coating | N | N | N | N | N | N | N | N | N | N |
| soak thru to | | | | | | | | | | |
| noncoated | | | | | | | | | | |
| side? (Y/N) | | | | | | | | | | |

⁽¹⁾polyethylene glycol - 600 Mw, Aldrich Chemical Co.
⁽²⁾polyethylene glycol - 1500 Mw, Aldrich Chemical Co.
⁽³⁾polyethylene glycol - 2000 Mw, Aldrich Chemical Co.
⁽⁴⁾hexylene glycol - 1,6-hexanediol, Aldrich Chemical Co.
⁽⁵⁾3-methoxy 3-methyl 1-butanol - Kuraray Chemical Co.

### Examples 20-25

Multi-functional facial wipes were prepared as in Examples 10-19 above except a 40,000 Mw PVP-2 was used as the film-forming polymer. Several different glycols were used at various percent solids to demonstrate possible variations in coating compositions as shown in Table 3 below.

TABLE 3

| | Example | | | | | |
|---|---|---|---|---|---|---|
| Component | 20 | 21 | 22 | 23 | 24 | 25 |
| PVP-2 | 46 | 46 | 46 | 46 | 46 | 46 |
| PPG-1⁽¹⁾ | 6 | | | | | |
| PEG-1 | | 6 | | | | |
| PEG-2 | | | 6 | | | |
| PPG-2⁽²⁾ | | | | 6 | | |
| HG-1 | | | | | 6 | |
| MMBG-1 | | | | | | 6 |
| Talc | 46 | 46 | 46 | 46 | 46 | 46 |
| Salicylic | 2 | 2 | 2 | 2 | 2 | 2 |
| acid | | | | | | |
| % Solids | 60 | 60 | 60 | 65 | 60 | 60 |
| Viscosity | 3750 | 3500 | — | 15000 | 3750 | 3250 |
| % | 46 | 46 | 46 | 46 | 46 | 46 |
| Particulates | | | | | | |
| after drying | | | | | | |
| Did coating | N | N | N | N | N | N |
| soak thru to | | | | | | |
| noncoated | | | | | | |
| side? (Y/N) | | | | | | |

⁽¹⁾polypropylene glycol - 400 Mw; Matheson, Coleman & Bell Chemical Co.
⁽²⁾polypropylene glycol - 2000 Mw, Aldrich Chemical Co.

### Examples 26-35

Multi-functional facial wipes were prepared as in Examples 20-25 above except several different ingredients were used in place of salicylic acid to demonstrate possible variations in coating compositions as shown in Table 4 below.

TABLE 4

| | Example | | | | | | | | | |
|---|---|---|---|---|---|---|---|---|---|---|
| Component | 26 | 27 | 28 | 29 | 30 | 31 | 32 | 33 | 34 | 35 |
| PVP-2 | 46 | | 46 | | 46 | | 46 | | 46 | |
| PVP-3 | | 46 | | 46 | | 46 | | 46 | | 46 |
| Butylene | 6 | 6 | 6 | 6 | 6 | 6 | 6 | 6 | | |
| glycol | | | | | | | | | | |
| Talc | 46 | 46 | 46 | 46 | 46 | 46 | 46 | 46 | 46 | 46 |
| Aloe gel⁽¹⁾ | 2 | 2 | | | | | | | | |
| Lanolin(2) | | | 2 | 2 | | | | | | |
| Almond | | | | | 2 | 2 | | | | |
| oil⁽³⁾ | | | | | | | | | | |
| Ascorbic | | | | | | | 2 | 2 | | |
| acid⁽⁴⁾ | | | | | | | | | | |
| Glitter(5) | | | | | | | | | 2 | 2 |
| % Solids | 65 | 65 | 65 | 65 | 65 | 65 | 65 | 65 | 65 | 65 |
| Viscosity | 9500 | 13250 | 13750 | 16250 | 12750 | 15300 | | 23750 | 12250 | 17250 |
| % | 46 | 46 | 46 | 46 | 46 | 46 | 46 | 46 | 48 | 48 |
| Particulates | | | | | | | | | | |
| after drying | | | | | | | | | | |
| Did coating | N | N | N | N | N | N | N | N | N | N |
| soak thru to | | | | | | | | | | |
| noncoated | | | | | | | | | | |
| side? (Y/N) | | | | | | | | | | |

⁽¹⁾AloeVera 80 - Naturade
⁽²⁾Lanolin - Fisher Scientific Co.
⁽³⁾Almond oil - Hain Pure Food Co.
⁽⁴⁾Ascorbic acid - Mallinckrodt Chemical Co.
⁽⁵⁾Ultrabrite glitter .015 Hex - Glitterex Corp.

### Examples 36-37

Multi-functional facial wipes were prepared as in Examples 26-35 above except aspirin (acetylsalicylic acid) and zinc oxide were used as ingredients in combination with a 10,000 Mw PVP to demonstrate possible variations in coating compositions as shown in Table 5 below.

TABLE 5

| | Example | |
|---|---|---|
| Component | 36 | 37 |
| PVP-1 | 46 | 46 |
| Butylene glycol | 6 | 6 |
| Talc | 46 | 46 |
| Aspirin⁽¹⁾ | 2 | |
| Zinc oxide⁽²⁾ | | 2 |
| % Solids | 75 | 75 |
| Viscosity | | |
| % Particulates after drying | 46 | 48 |
| Did coating soak thru to | N | N |
| noncoated side? (Y/N) | | |

⁽¹⁾acetylsalicylic acid - commercial aspirin
⁽²⁾zinc oxide - Aldrich Chemical Co.

### Examples 38-46

Multi-functional facial wipes were prepared as in Examples 1-9 above except different film-forming polymers were used in place of polyvinylpyrrolidone to demonstrate possible variations in coating compositions as shown in Table 6 below.

TABLE 6

| | Example | | | | | | | | |
|---|---|---|---|---|---|---|---|---|---|
| Component | 38 | 39 | 40 | 41 | 42 | 43 | 44 | 45 | 46 |
| QVP⁽¹⁾ | 46 | 46.7 | | 32.2 | | | | | |
| PVP-1 | | | | | 10 | 15 | 20 | 26 | 26 |
| PEI⁽²⁾ | | | | | 36 | 31 | 26 | 18 | 15 |
| PVP/VA⁽³⁾ | | | 46 | 24.2 | | | | | |
| Butylene | 6 | 5 | 6 | | 6 | 6 | 6 | 6 | 6 |
| glycol | | | | | | | | | |
| Pharma- | | | | 1.7 | | | | | |
| Solve⁽⁴⁾ | | | | | | | | | |
| Talc | 46 | 46.7 | 46 | 40.2 | 46 | 46 | 46 | 46 | 46 |
| Salicylic | 2 | 1.7 | 2 | | 2 | 2 | 2 | 2 | 2 |
| acid | | | | | | | | | |
| Span 20⁽⁵⁾ | | | | | | | | 2 | |
| Glycerol⁽⁶⁾ | | | | | | | | | 5 |
| % Solids | 70 | 50 | 60 | 50 | 78 | 78 | 78 | 78 | 78 |
| Viscosity | 3250 | 3630 | 1720 | | | | | | |
| % | 46 | 46.7 | 46 | 40.2 | 46 | 46 | 46 | 46 | 46 |
| Particulates | | | | | | | | | |
| after drying | | | | | | | | | |
| Did coating | N | N | N | N | N | N | N | N | N |
| soak thru to | | | | | | | | | |
| noncoated | | | | | | | | | |
| side? (Y/N) | | | | | | | | | |

⁽¹⁾GAFQUAT 734 - Quarternized vinylpyrrolidone/Dimethylaminoethylmethaclylate copolymer - 100,000 Mw, 50% ethanol solution, ISP Technologies Inc.
⁽²⁾polyethylenimine - 750,000 Mw, 50% aqueous solution, Aldrich Chemical Co.
⁽³⁾Plasdone S-630 - vinylacetate/N-vinylpyrrolidone copolymer, ISP Technologies Inc.
⁽⁴⁾N-methyl-2-pyrrolidone, ISP Technologies
⁽⁵⁾Span 20- sorbitan monolaurate, Ruger Chemical Co. Inc.
⁽⁶⁾Glycerol, Mallinckrodt Inc.

### Comparative Examples C1-C4

The compositions of Examples 3-6 were coated onto two commercially available facial oil removing paper tissues available from the Kose Co. and Yojiya Co. of Japan using the same coating technique as in the above examples. The results in Table 7 below show that at low percent solids and viscosities, the coatings soaked through the tissues and were visible on the uncoated side of the tissue papers.

TABLE 7

| | Counter Examples | | | |
|---|---|---|---|---|
| | Paper Tissue 1⁽¹⁾ | | Paper Tissue 2⁽²⁾ | |
| Component | C1 | C2 | C3 | C4 |
| PVP-2 | 46 | 46 | 46 | 46 |
| Butylene glycol | 6 | 6 | 6 | 6 |
| Talc | 46 | 46 | 46 | 46 |
| Salicylic acid | 2 | 2 | 2 | 2 |
| % Solids | 60 | 65 | 60 | 65 |
| Viscosity | 3500 | 13250 | 3500 | 13250 |
| % Particulates | 46 | 46 | 46 | 46 |
| after drying | | | | |
| Did coating | Y | Y | Y | Y |
| soak thru to | | | | |
| noncoated side? | | | | |
| (Y/N) | | | | |

⁽¹⁾facial blotting tissue - Kose Co. of Japan
⁽²⁾facial blotting tissue - Yojiya Co. of Japan

### Examples 47-50

The compositions used in Table 7 above were coated onto the same paper tissues as in C1-C4 except the viscosity of the compositions was increased by increasing the percent solids. The results in Table 8 below show that at higher percent solids and viscosities, the coatings do not soak entirely through the paper tissues, although the coatings were still visible on the uncoated side of the tissue papers.

TABLE 8

| | Examples | | | |
|---|---|---|---|---|
| | Paper Tissue 1 | | Paper Tissue 2 | |
| Component | 47 | 48 | 49 | 50 |
| PVP-2 | 46 | 46 | 46 | 46 |
| Butylene glycol | 6 | 6 | 6 | 6 |
| Talc | 46 | 46 | 46 | 46 |
| Salicylic acid | 2 | 2 | 2 | 2 |
| % Solids | 70 | 75 | 70 | 75 |
| Viscosity | 37750 | 274000⁽¹⁾ | 37750 | 274000⁽¹⁾ |
| % Particulates | 46 | 46 | 46 | 46 |
| after drying | | | | |
| Did coating | N | N | N | N |
| soak thru to | | | | |
| noncoated side? | | | | |
| (Y/N) | | | | |

⁽¹⁾spindle 4 at 1.5 RPM

### Comparative Examples C5-C9

It is well known in the patent art to impregnate tissues and nonwovens with medicated, cleansing, or cosmetic compositions. Several compositions from the wipe patent art were selected and coated onto the microporous film of Examples 1-46 using the same coating technique as in the above examples. The results in Table 9 below show that known compositions from the patent art do not work when coated onto the microporous films of the invention because they soak through the film and are visible from the uncoated side, or the coatings are invisible on the coated side, or are extremely stiff after drying. C9 was prepared by taking the composition of Examples 1 and 2 and diluting it to 60% solids to demonstrate that low viscosity coatings soak through the film.

TABLE 9

| | Comparative example | | | | |
|---|---|---|---|---|---|
| Component | C5^{(a)} | C6^{(b)} | C7^{(c)} | C8^{(d)} | C9^{(e)} |
| PVA⁽¹⁾ | | 9.5 | 9.5 | | 46 |
| Glycerol⁽²⁾ | | 19.0 | 19.0 | | |
| Butylene glycol | | | | | 6 |
| Petrolatum⁽³⁾ | 34.6 | | | | |
| Salicylic acid | | | | 1.5 | |
| Citric acid⁽⁴⁾ | 20.1 | | | | |
| Propanediol⁽⁵⁾ | 10.0 | | | | |
| Talc | | | 42.9 | | 46 |
| Kaolin clay⁽⁶⁾ | | 42.9 | | | |
| Hexadecanol⁽⁷⁾ | 10.1 | | | | |
| Octadecanol⁽⁸⁾ | 25.2 | | | | |
| Ethanol⁽⁹⁾ | | | | 25.4 | |
| Distilled water | | 28.6 | 28.6 | 72.7 | |
| Aloe Vera⁽¹⁰⁾ | | | | 0.4 | |
| % Solids | 54.7 | 71.4 | 71.4 | 1.9 | 60 |
| Viscosity | | | | | 375 |
| % Particulates | | 60 | 60 | 100 | 46 |
| after drying | | | | | |
| Did coating | Y | N | N | N | Y |
| soak thru to | | coating | coating | coating | |
| noncoated side? | | was | was | was not | |
| (Y/N) | | brittle | brittle | visible | |

^{(a)}U.S. Pat. 5,830,487 Example 1
^{(b)}U.S. Pat. 4,569,343 Example 1 lines 55-60
^{(c)}Same as (b) except talc was substituted for clay
^{(d)}U.S. Pat. 5,744,149 Example VI
^{(e)}Example 1 except 60% solids
⁽¹⁾Polyvinyl alcohol 10,000 Mw - Aldrich Chemical Co.
⁽²⁾Gycerol- Mallinckrodt Inc
⁽³⁾Petrolatum - E.M. Science
⁽⁴⁾Citric acid - E.M. Science
⁽⁵⁾Propanediol - Aldrich Chemical Co.
⁽⁶⁾Kaolin clay - Aldrich Chemical Co.
⁽⁷⁾Hexadecanol - Aldrich Chemical Co.
⁽⁸⁾Octadecanol - Aldrich Chemical Co.
⁽⁹⁾Ethanol - E.M. Science
⁽¹⁰⁾Aloe Vera - Fruit of the Earth Inc.

### Examples 51-54

To demonstrate the effect of the coatings of the invention on other microporous films, the same composition and coating technique used in Example 2 was applied to four commercially available microporous films(MPF) as shown in Table 10.

TABLE 10

| | Example | | | |
|---|---|---|---|---|
| Component | 51 | 52 | 53 | 54 |
| Film type | MPF-1⁽¹⁾ | MPF-2⁽²⁾ | MPF-3⁽³⁾ | MPF-4⁽⁴⁾ |
| PVP-1 | 46 | 46 | 46 | 46 |
| Butylene glycol | 6 | 6 | 6 | 6 |
| Talc | 46 | 46 | 46 | 46 |
| Salicylic acid | 2 | 2 | 2 | 2 |
| % solids | 75 | 75 | 75 | 75 |
| Viscosity | 22520 | 22500 | 22500 | 22500 |
| % Particulates | 46 | 46 | 46 | 46 |
| after drying | | | | |
| Did coating | N | N | N | N |
| soak thru to | | | | |
| noncoated side? | | | | |
| (Y/N) | | | | |

⁽¹⁾AP-3 particle-filled polypropylene microporous film - Amoco Films and Fabrics
⁽²⁾Exxaire particle-filled polyethylene microporous film, 35 GSM - Exxon
⁽³⁾Particle-filled polyethylene microporous film - Kimberly Clark Corp.
⁽⁴⁾Celgard 2400 polypropylene microporous film - Celgard Inc.

Example 55 and Comparative Examples C10-C12

To demonstrate the effect of the coatings of the invention on an alternative porous wipe, the same composition and coating technique used in Example 2 was applied to a consolidated nonwoven web formed of polypropylene microfibers. The results in Table 11 below show that at lower percent solids and viscosities the coatings soak through the wipe, but by increasing the viscosity, coatings can be prepared that adequately anchor to the wipe but do not penetrate all the way through to the uncoated side so as to interfere with the intended functionality of the wipe. The consolidated nonwoven web was prepared using apparatus similar to that shown in FIG. 1 of the drawings. Fina 3960, a 350 melt flow index polypropylene resin, was fed into the extruder 13, the temperature of the die 10 was maintained at 371° C., the attenuating air was delivered to the die at a temperature of 390° C. and a flow rate of 5.3 cubic meters per minute. The polypropylene was delivered to the die at a rate of 0.20 kg/hr/cm. The basis weight of the web was 21 grams/m². The web was then calendered by passing the web, at 15.2 meters/minute, through a nip formed by an upper heated smooth steel roll 24 and a lower unheated 95 Shore A hard rubber roll 25. The nip pressure was 1050 Newtons per lineal centimeter. The temperature of the upper steel roll was 88° C. The caliper of the calendered web was 72 microns.

TABLE 11

| | Consolidated Nonwoven | | | |
|---|---|---|---|---|
| Component | C10 | C11 | C12 | 55 |
| PVP-2 | 46 | 46 | 46 | 46 |
| Butylene | 6 | 6 | 6 | 6 |
| glycol | | | | |
| Talc | 46 | 46 | 46 | 46 |
| Salicylic | 2 | 2 | 2 | 2 |
| acid | | | | |
| % Solids | 60 | 65 | 70 | 75 |
| Viscosity | 3500 | 13250 | 37750 | 274000⁽¹⁾ |
| % | 46 | 46 | 46 | 46 |
| Particulates | | | | |
| after drying | | | | |
| Did coating | Y | Y | Y | N |
| soak thru to | | | | |
| noncoated | | | | |
| side? (Y/N) | | | | |

⁽¹⁾spindle 4 at 1.5 RPM

To demonstrate the effect of filler content on the compositions of the invention, talc was mixed with PVP at various loadings and diluted to 50% solids in isopropyl alcohol. The solutions were then dried and observations made as to the physical nature of the coating. When the percent particulates of the coating compositions exceeds 40% the coating becomes brittle and has a tendency to flake off of the wipe substrate. Results are reported in Table 12 below.

TABLE 12

| % Talc | % PVP⁽¹⁾ | Comments |
|---|---|---|
| 0 | 100 | Rubbery, tacky |
| 20 | 80 | Rubbery, tacky |
| 40 | 60 | Tough, not tacky |
| 60 | 40 | Brittle, not tacky |
| 80 | 20 | Very brittle, not tacky |
| 100 | 0 | Powdery, not tacky |

⁽¹⁾polyvinylpyrrolidone - Povidone K-30, 40,000 Mw, BASF Chemical Co.

We claim:

1. An oil absorbing wipe material suitable for wiping a users skin comprising an oil absorbing porous substrate having two faces wherein the substrate has a transparency of less than 65 percent which porous substrate changes transparency upon absorption of oil, said porous substrate having a generally non-tacky flexible coating on at least a portion of at least one face, said coating comprising a film forming polymer with at least one additional additive which coating is visible on the coated face of the porous substrate and which coating does not penetrate to the opposite face of the porous substrate.

2. The oil absorbing wipe material of claim 1 wherein the oil absorbing wipe is a filmlike thermoplastic material.

3. The oil absorbing wipe material of claim 1 wherein the oil absorbing wipe is a consolidated oil absorbing paper wipe.

4. The oil absorbing wipe material of claim 1 wherein the coating comprises at least a film forming polymer and a particulate filler.

5. The oil absorbing wipe material of claim 4 wherein the particulate filler comprises 35 to 55 percent by weight of the coating and has an average particle size of from 0.1 to 30 microns.

6. The oil absorbing wipe material of claim 5 wherein the film forming polymer is at lease a partially water soluble film forming polymer.

7. The oil absorbing wipe material of claim 5 wherein the film forming polymer is at least a partially water insoluble film forming polymer.

8. The oil absorbing wipe material of claim 1 wherein the film forming polymer coating penetrates from 10 to 90 percent of the thickness of the oil absorbing porous substrate.

9. The oil absorbing wipe material of claim 8 wherein the film forming polymer coating penetrates from 20 to 80 percent of the thickness of the oil absorbing porous substrate.

10. The oil absorbing wipe material of claim 1 wherein the additional film forming polymer comprises polyvinylpyrrolidone.

11. The oil absorbing wipe material of claim 1 wherein the additional additive is an active or skin modifying agent.

12. The oil absorbing wipe material of claim 11 wherein the active or skin modifying agent is salicylic acid.

13. The oil absorbing wipe material of claim 1 wherein the coating additive comprises nonactive agents.

14. The oil absorbing wipe material of claim 1 wherein the coating further comprises a gelling agent.

15. The oil absorbing wipe material of claim 1 wherein the coating further comprises a filler.

16. The oil absorbing wipe material of claim 8 wherein the oil absorbing porous sheet comprises a porous stretched film made of a thermoplastic material.

17. The oil absorbing wipe material of claim 16 wherein interstitial volume per unit area of said porous stretched film is in the range of 0.0001-0.005 cm³ as calculated by the following equation:
interstitial volume per unit area=[film thickness (cm)×1 (cm)×void content (%)]/100(where the void content is the percentage of voids in the porous film).

18. The oil absorbing wipe material of claim 16 wherein the void content of said porous stretched film is in the range of 5-50% and the film thickness is in the range of 5-200 μm.

19. The oil absorbing wipe material of claim 16 wherein the porous film comprises a thermoplastic polymer film having from 20 to 60 percent by weight of a filler.

20. The oil absorbing wipe material of claim 19 wherein the porous film contains a non-particulate filler.

21. The oil absorbing wipe material of claim 20 wherein the non-particulate filler is mineral oil.

22. The oil absorbing wipe material of claim 16 wherein the porous film voids have an average size is in the range of from 0.2 to 5.0 microns (μm).

23. The oil absorbing wipe material of claim 17 wherein the interstitial volume per unit area is from 0.0002 to 0.001 cm³.

24. The oil absorbing wipe material of claim 1 wherein the porous oil absorbing wipe comprises a consolidated melt-blown web of thermoplastic fibers.

25. The oil absorbing wipe material of claim 1 wherein the porous oil absorbing sheet has an opacity value of about 65 or less when oil free, and which web changes transparency by at least 30 percentage points when loaded with about 6 grams or less of oil per square centimeter.

26. The oil absorbing wipe material claim 24 wherein the thermoplastic fibers are polyolefin microfibers.

27. The oil absorbing wipe material of claim 24 wherein the thermoplastic fibers are polypropylene microfibers.

28. The oil absorbing wipe material of claim 24 wherein the thermoplastic fibers have an average diameter of about 10 micrometers or less, and the wipe has a basis weight of about 40 gm/m² or less.

29. The oil absorbing wipe material of claim 25 wherein the wipe, when it has changed transparency, has a transparency of about 90 or greater.

30. The oil absorbing wipe material of claim 25 wherein the web changes in transparency by 35 or more when loaded with about 6 grams or less of oil per square meter.

31. The oil absorbing wipe material of claim 24 wherein the wipes have a void volume of from 40 to 80 percent.

32. The oil absorbing wipe material of claim 24 wherein the wipes have a void volume of from 45 to 75 percent.

33. The oil absorbing wipe material of claim 24 wherein the wipes have a void volume of from 50 to 70 percent.

34. The oil absorbing wipe material of claim 24 wherein the average pore size of the wipe material is from 3 to 15 microns.

35. The oil absorbing wipe material of claim 24 wherein the average pore size of the wipe material is from 3 to 12 microns.

36. The oil absorbing wipe material of claim 24 wherein the average pore size of the wipe material is from 4 to 8 microns.

37. The oil absorbing wipe material of claim 24 wherein the wipes have an oil absorption capacity of from 0.7 to 6 mg/cm².

38. The oil absorbing wipe material of claim 24 wherein the wipes have a basis weight of from 10 to 30 gm/m².

39. The oil absorbing wipe material of claim 1 wherein the wipes have a Hand of 8 grams or less.

40. The oil absorbing wipe material claim 1 wherein the wipes have a Hand of 1 to 6 grams or less.

41. The oil absorbing wipe material of claim 1 wherein the coating is uniformly provided on at least a portion of one face of the oil absorbing wipe product.

42. The oil absorbing wipe material of claim 41 wherein the coating is provided on from 50 to 100 percent of one face of the oil absorbing wipe.

43. The oil absorbing wipe material of claim 42 wherein the coating is continuous.

44. The oil absorbing wipe material of claim 42 wherein the coating is pattern coating.

45. A method for forming a flexible coating on an oil absorbing wipe material suitable for wiping a users skin comprising providing an oil absorbing porous substrate having two faces wherein the substrate has a transparency of less than 65 percent which porous substrate changes transparency upon absorption of oil, coating the porous substrate on at least a portion of at least one face with a coating solution comprising at least a film forming polymer, a particulate filler and an evaporative solvent with at least one additional additive, the coating solution having a viscosity of from 2000 to 100,000 cps and a percent solids of 60 to 80 percent wherein the coating is visible on the coated face of the porous substrate and which coating does not penetrate to the opposite face of the porous substrate.

46. The method of forming an oil absorbing wipe material of claim 45 wherein the oil absorbing wipe is a film-like thermoplastic material and the coating solution has a viscosity of from 3000 to 50,000.

47. The method of forming an oil absorbing wipe material of claim 45 wherein the oil absorbing wipe is a consolidated oil absorbing paper wipe and the coating solution has a viscosity of from 10,000 to 100,000.

48. The method of forming an oil absorbing wipe material of claim 45 wherein the coating comprises at least a film forming polymer and a particulate filler such that the dried coating has 35 to 55 percent particulate filler to other nonparticulate solids said filler having an average particle size of from 0.1 to 30 microns.

49. The method of forming an oil absorbing wipe material of claim 48 wherein the particulate filler comprises 40 to 50 percent by weight of the solids.

50. The method of forming an oil absorbing wipe material of claim 49 wherein the film forming polymer is at least a partially water soluble film forming polymer.

51. The method of forming an oil absorbing wipe material of claim 49 wherein the film forming polymer coating is at least a partially water insoluble film forming polymer.

52. The method of forming an oil absorbing wipe material of claim 45 wherein the film forming polymer coating penetrates from 10 to 90 percent of the thickness of the oil absorbing porous substrate.

53. The method of forming an oil absorbing wipe material of claim 52 wherein the film forming polymer coating penetrates from 20 to 80 percent of the thickness of the oil absorbing porous substrate.

54. The method of forming an oil absorbing wipe material of claim 45 wherein the film forming polymer comprises polyvinylpyrrolidone.

55. The method of forming an oil absorbing wipe material of claim 45 wherein the additional additive is an active or skin modifying agent.

56. The method of forming an oil absorbing wipe material of claim 55 wherein the active or skin modifying agent is salicylic acid.

57. The method of forming an oil absorbing wipe material of claim 45 wherein the coating further comprises a gelling agent.

58. The method of forming an oil absorbing wipe material of claim 45 wherein the coating is uniformly provided on at least a portion of one face of the oil absorbing wipe product.

59. The method of forming an oil absorbing wipe material of claim 58 wherein the coating is provided on from 50 to 100 percent of one face of the oil absorbing wipe.

60. The method of forming an oil absorbing wipe material of claim 59 wherein the coating is continuous.

61. The method of forming an oil absorbing wipe material of claim 59 wherein the coating is a pattern coating.
